(19) United States
(12) Patent Application Publication
(10) Pub. No.: US 2024/0138738 A1
(43) Pub. Date: May 2, 2024

(54) BIOPOTENTIAL ELECTRODE BODY AND BIOPOTENTIAL SENSOR

(71) Applicant: SONY GROUP CORPORATION, Tokyo (JP)

(72) Inventors: Mao KATSUHARA, Tokyo (JP); Ryo SASAKI, Tokyo (JP)

(73) Assignee: SONY GROUP CORPORATION, Tokyo (JP)

(21) Appl. No.: 18/548,656

(22) PCT Filed: Mar. 2, 2022
(86) PCT No.: PCT/JP2022/008878

§ 371 Date: Sep. 1, 2023

(30) Foreign Application Priority Data
Mar. 11, 2021 (JP) 2021-039637
Feb. 14, 2022 (JP) 2022-020872

(51) Int. Cl. A61B 5/291; A61B 5/053; A61B 5/265; A61B 5/268; A61B 5/31

(52) U.S. Cl. CPC A61B 5/291; A61B 5/053; A61B 5/265; A61B 5/268; A61B 5/31; A61B 2560/02; A61B 2560/0468; A61B 2562/0215

(57) ABSTRACT

Provided is a biopotential electrode body having high mechanical strength and suitable for acquiring more bioinformation. The biopotential electrode body includes multiple electrically conductive portions each having a contact surface contactable with a biological body, and one or more electrically insulating portions that electrically insulate the multiple electrically conductive portions from each other. The electrically conductive portions each include a first elastic resin and a plurality of first particles buried in the first elastic resin. The electrically insulating portions each include a second elastic resin and a plurality of second particles buried in the second elastic resin.

### TECHNICAL FIELD

The present disclosure relates to a biopotential sensor that is allowed to come into contact with a biological body to detect a biological signal, and a biopotential electrode body used for the biopotential sensor.

### BACKGROUND ART

Earphone-type biopotential sensors that detect biological signals such as brain waves, heart rates, or pulses have been proposed (for example, see PTL 1).

### CITATION LIST

### Patent Literature

PTL 1: Japanese Unexamined Patent Application Publication No. 2019-24758

### SUMMARY OF THE INVENTION

In PTL 1, an earpiece portion to come into contact with an external ear includes a conductive material so that the earpiece portion serves as an electrical contact with a biological body.

Meanwhile, it is desirable that a biopotential sensor be superior in durability and be able to acquire more bioinformation.

It has therefore been demanded to provide a biopotential sensor having high mechanical strength and suitable for acquiring more bioinformation, and a biopotential electrode used for the biopotential sensor.

A biopotential electrode body according to an embodiment of the present disclosure includes multiple electrically conductive portions each having a contact surface contactable with a biological body, and one or more electrically insulating portions that electrically insulate the multiple electrically conductive portions from each other. The electrically conductive portions each include a first elastic resin and a plurality of first particles buried in the first elastic resin. The electrically insulating portions each include a second elastic resin and a plurality of second particles buried in the second elastic resin.

A biopotential sensor according to an embodiment of the present disclosure includes the biopotential electrode body described above.

The biopotential electrode body and the biopotential sensor according to the embodiments of the present disclosure each include the multiple electrically conductive portions insulated from each other. This makes it possible to detect electric potential at a plurality of locations in a biological body. Furthermore, the electrically conductive portions each include the plurality of first particles, and the electrically insulating portions each include the plurality of second particles. This helps to prevent the electrically conductive portions and the electrically insulating portions from peeling off.

### BRIEF DESCRIPTION OF DRAWINGS

FIG. 1 is a cross-sectional view schematically illustrating an overall configuration of an electrode body unit including a pair of electrode bodies according to a first embodiment of the present disclosure.

FIG. 2 is a perspective view schematically illustrating an appearance of either one of the electrode bodies illustrated in FIG. 1.

FIG. 3 is a cross-sectional view schematically illustrating another cross-section of either one of the electrode bodies illustrated in FIG. 1.

FIG. 4 is a cross-sectional view illustrating in detail the one of the electrode bodies illustrated in FIG. 1.

FIG. 5 is a cross-sectional view illustrating details of one first particle included in either one of the electrode bodies illustrated in FIG. 1.

FIG. 6 is a cross-sectional view illustrating details of another first particle included in either one of the electrode bodies illustrated in FIG. 1.

FIG. 7 is a cross-sectional view illustrating a configuration example of an electrode body according to a first modification example to the first embodiment of the present disclosure.

FIG. 8 is a cross-sectional view illustrating a configuration example of an electrode body according to a second modification example to the first embodiment of the present disclosure.

FIG. 9A is a perspective view schematically illustrating an overall configuration example of a biopotential sensor according to a second embodiment of the present disclosure.

FIG. 9B is an exploded perspective view of the biopotential sensor illustrated in FIG. 9A.

FIG. 10 is a block diagram schematically illustrating the overall configuration example of the biopotential sensor illustrated in FIG. 9A.

FIG. 11 is a cross-sectional view illustrating configuration examples of electrode bodies according to a first modification example to the second embodiment of the present disclosure.

FIG. 12 is a cross-sectional view illustrating a configuration example of an electrode body according to a second modification example to the second embodiment of the present disclosure.

FIG. 13 is a block diagram schematically illustrating an overall configuration example of a biopotential sensor according to a third modification example to the second embodiment of the present disclosure.

FIG. 14 is a block diagram schematically illustrating an overall configuration example of a biopotential sensor according to a fourth modification example to the second embodiment of the present disclosure.

FIG. 15 is a schematic diagram illustrating an example where the electrode bodies illustrated in FIG. 1 are used in a head mount display.

FIG. 16 is a schematic diagram illustrating an example where the electrode bodies illustrated in FIG. 1 are used in a headband.

FIG. 17 is a schematic diagram illustrating an example where the electrode bodies illustrated in FIG. 1 are used in a headphone.

FIG. 18 is a schematic diagram illustrating an example where the electrode bodies illustrated in FIG. 1 are used in a cap.

### MODES FOR CARRYING OUT THE INVENTION

Some embodiments of the present disclosure will now be described in detail with reference to the accompanying drawings. The embodiments described below are specific, but mere examples of the present disclosure. The technology according to the present disclosure is not limited to those configurations described below. The arrangements, sizes, dimensional ratios, and other factors of components in the present disclosure are not also limited to those configurations illustrated in the drawings.

Note that the description is given in the following order.
0. Circumstances
1. First Embodiment (Biopotential Electrode Body)
1.1. Configuration Example
1.2. Manufacturing Method
1.3. Workings and Effects
1.4. Modification Examples
2. Second Embodiment (Biopotential Sensor)
2.1. Configuration Example
2.2. Workings and Effects
2.3. Modification Examples
3. Other Modification Examples

### 0. Circumstances

Wet electrodes have been used so far to measure biopotential such as brain waves or bioelectrical impedance such as electrodermal activities. Wet electrodes reduce contact impedance with a biological body when a gel or a physiological saline solution for measurement purposes are used, for example. However, the use of such wet electrodes has been avoided in many consumer applications in view of uncleanliness due to adhesion of the gel, a change in the gel over time, or inconvenience of preparing for measurements, for example. Measuring instruments including dry electrodes therefore begin to be proposed in these days (see “Dry EEG Electrodes” Sensors, 14, 12847 (2014), for example).

Various materials for such dry electrodes have also been proposed. For example, Japanese Unexamined Patent Application Publication No. 2010-247047 proposes a nitrile rubber composition in which electrically conductive particles such as carbon particles are mixed with an elastomer material such as rubber. Since the nitrile rubber composition is rich in flexibility, using such a nitrile rubber composition as a material for dry electrodes is expected to improve comfortability during attachment.

Meanwhile, such efforts have been taken in recent years that allows human bodies to undergo electrical measurements to acquire useful information from the human bodies (for example, see “Hearables: Multimodal physiological in-ear sensing” Scientific Reports, 7, 6948(2017), and “Unobtrusive ambulatory EEG using a smartphone and flexible printed electrodes around the ear” Scientific Reports, 5, 16743(2015)). In that case, the electrode is allowed to come into contact with a skin surface of a limited portion of a human body, such as an external ear or a peripheral portion of the external ear of the human body, for example.

In PTL 1 described above, for example, an electrically conductive resin is used at an electrical contact with an inner surface of an external acoustic pore. In the earphone-type biopotential sensor described in PTL 1, however, the electrode is allowed to come into contact with a skin surface of a limited portion such as the inner surface of the external acoustic pore. Bioinformation is therefore acquired only from the limited portion.

To solve such an issue, the present disclosure provides a biopotential sensor suitable for acquiring more bioinformation, and a biopotential electrode body used for the biopotential sensor.

### 1. First Embodiment

### 1.1. Configuration Example

### (Electrode Body Unit 1)

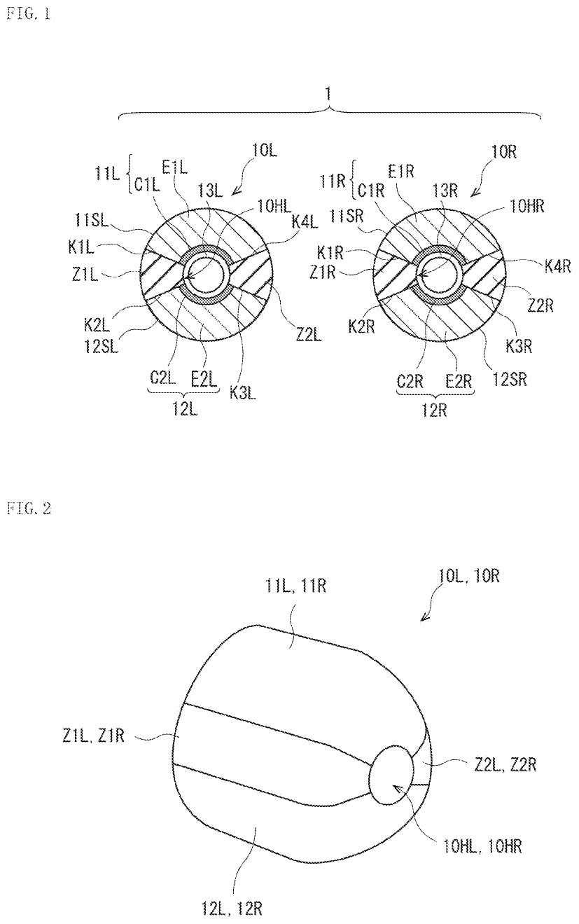

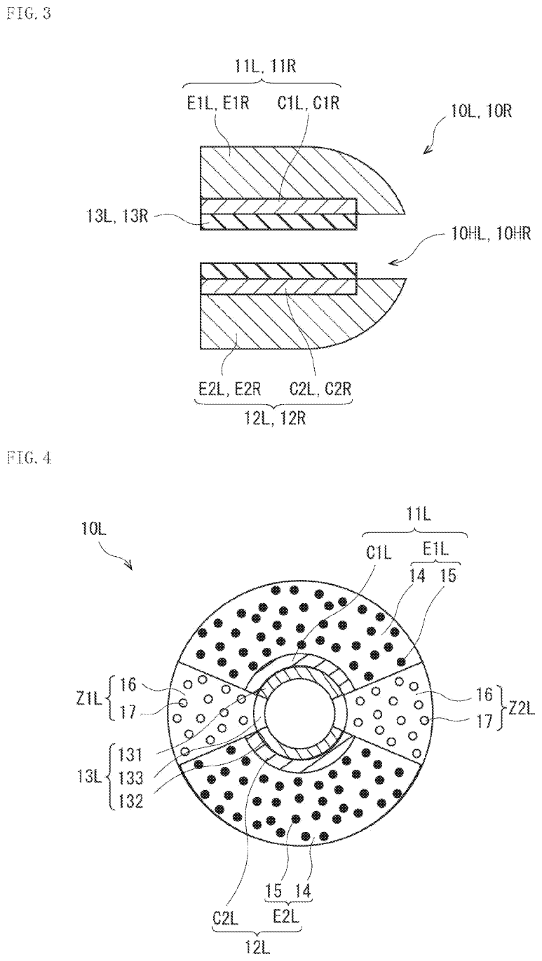

First, an electrode body unit 1 that corresponds to a biopotential electrode body according to a first embodiment of the present disclosure will now be described with reference to FIGS. 1 to 3. FIG. 1 is a first cross-sectional view schematically illustrating an overall configuration example of the electrode body unit 1. FIG. 2 is a perspective view schematically illustrating an appearance of either one of electrode bodies 10L and 10R. FIG. 3 is a second cross-sectional view illustrating a cross-section orthogonal to the cross-sections of the electrode bodies 10L and 10R illustrated in FIG. 1.

As illustrated in FIG. 1, the electrode body unit 1 includes the paired electrode bodies 10L and 10R. As illustrated in FIGS. 1 to 3, the paired electrode bodies 10L and 10R have respectively structures substantially identical to each other. Description is therefore given below of one of the paired electrode bodies 10L and 10R, i.e., the electrode body 10L. Note that, when it is not necessary to distinguish the paired electrode bodies 10L and 10R from each other, each of the electrode bodies 10L and 10R is herein simply referred to as an electrode body 10 as appropriate.

### (Electrode Body 10L)

The electrode body 10L includes multiple electrically conductive portions and one or more electrically insulating portions. Specifically, the multiple electrically conductive portions include a first electrically conductive portion 11L and a second electrically conductive portion 12L, for example. Specifically, the one or more electrically insulating portions include a first electrically insulating portion Z1L and a second electrically insulating portion Z2L, for example. The first electrically conductive portion 11L, the second electrically conductive portion 12L, the first electrically insulating portion Z1L, and the second electrically insulating portion Z2L form a cylindrical structure as a whole. The cylindrical structure has a substantially cylindrical shape having a through hole 10HL. The cylindrical structure formed by the first electrically conductive portion 11L, the second electrically conductive portion 12L, the first electrically insulating portion Z1L, and the second electrically insulating portion Z2L is supported by a support member 13L extending in the through hole 10HL, for example. The first electrically conductive portion 11L, the first electrically insulating portion Z1L, the second electrically conductive portion 12L, and the second electrically insulating portion Z2L are alternately disposed in a circumferential direction of the cylindrical structure described above. The first electrically conductive portion 11L and the first electrically insulating portion Z1L are coupled to each other at an interface K1L. The first electrically insulating portion Z1L and the second electrically conductive portion 12L are coupled to each other at an interface K2L. The second electrically conductive portion 12L and the second electrically insulating portion Z2L are coupled to each other at an interface K3L. The second electrically insulating portion Z2L and the first electrically conductive portion 11L are coupled to each other at an interface K4L. The first electrically conductive portion 11L and the second electrically conductive portion 12L are therefore electrically insulated from each other by the first electrically insulating portion Z1L and the second electrically insulating portion Z2L.

### (First Electrically Conductive Portion 11L)

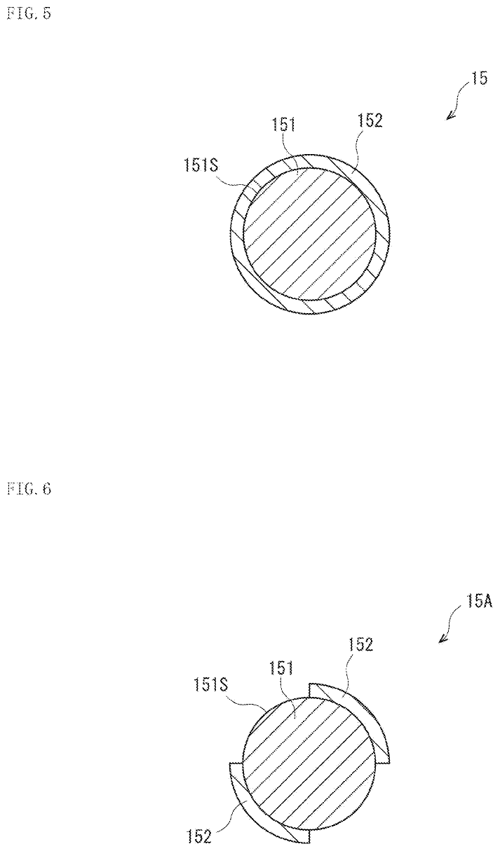

The first electrically conductive portion 11L has a two-layer structure including a first core portion C1L and a first elastic portion E1L. The first core portion C1L and the first elastic portion E1L are stacked on each other in a radial direction of the cylindrical structure. The first core portion C1L lies on an inner side of the cylindrical structure. The first elastic portion E1L lies on an outer side of the cylindrical structure. The first elastic portion E1L has a first surface 11SL that lies on an opposite side to the through hole 10HL. The first surface 11SL is a contact surface contactable with a biological body surface, such as a skin surface of a human body. The first core portion C1L includes a highly electrically conductive metal material, such as gold (Au), silver (Ag), or copper (Cu), as a main constituent material. The first core portion C1L is a portion in contact with a first electrode 131 of the support member 13L (see FIG. 4 to be described later) extending in the through hole 10HL, for example.

### (Second Electrically Conductive Portion 12L)

The second electrically conductive portion 12L is substantially identical in configuration to the first electrically conductive portion 11L described above. That is, the second electrically conductive portion 12L has a two-layer structure including a second core portion C2L and a second elastic portion E2L. The second core portion C2L and the second elastic portion E2L are stacked on each other in the radial direction of the cylindrical structure. The second core portion C2L lies on the inner side of the cylindrical structure. The second elastic portion E2L lies on the outer side of the cylindrical structure. The second elastic portion E2L has a second surface 12SL that lies on the opposite side to the through hole 10HL. The second surface 12SL is a contact surface contactable with a biological body surface, such as a skin surface of a human body. The second core portion C2L includes a highly electrically conductive metal material, such as gold (Au), silver (Ag), or copper (Cu), as a main constituent material. The second core portion C2L is a portion in contact with a second electrode 132 of the support member 13L (see FIG. 4 to be described later) extending in the through hole 10HL, for example.

### (First Elastic Portion E1L and Second Elastic Portion E2L)

FIG. 4 is a cross-sectional view illustrating details of the electrode body 10L illustrated in FIG. 1. As illustrated in FIG. 4, the first elastic portion E1L and the second elastic portion E2L each include a first elastic resin 14 and a plurality of first particles 15 buried in the first elastic resin 14. The first elastic resin 14 is an electrically insulating resin, for example. In contrast, the first particles 15 are electrically conductive particles, for example. Since the first particles 15 serving as electric conductors are included in a dispersed manner in the first elastic resin 14, the first elastic portion E1L and the second elastic portion E2L each have electric conductivity. The first elastic portion E1L and the second elastic portion E2L each include the first particles 15 at 30 wt %, for example.

It is preferable that an average primary particle diameter of the first particle 15 fall within a range from 10 nm to 100 nm both inclusive, for example. One reason for this is that a state in which the first particles 15 are more evenly dispersed in the first elastic resin 14 is easily attained. In a case where the first particles 15 are electrically conductive particles, dispersing the first particles 15 more evenly in the first elastic resin 14 is preferable because the electric conductivity of the first elastic portion E1L and the electric conductivity of the second elastic portion E2L are improved.

It is further preferable that an average secondary particle diameter of the first particle 15 fall within a range from 30 nm to 500 nm both inclusive. When a base particle 111 has a secondary particle diameter falling within the range described above, a state in which electrically conductive particles 103 are more easily dispersed in a base resin layer 101 is easily attained. This makes it possible to further improve measuring accuracy.

Here, the average primary particle diameter and the average secondary particle diameter of the base particle 111 may be measured through direct observation using a transmission electron microscope (TEM) or particle diameter measurement based on a dynamic light scattering method.

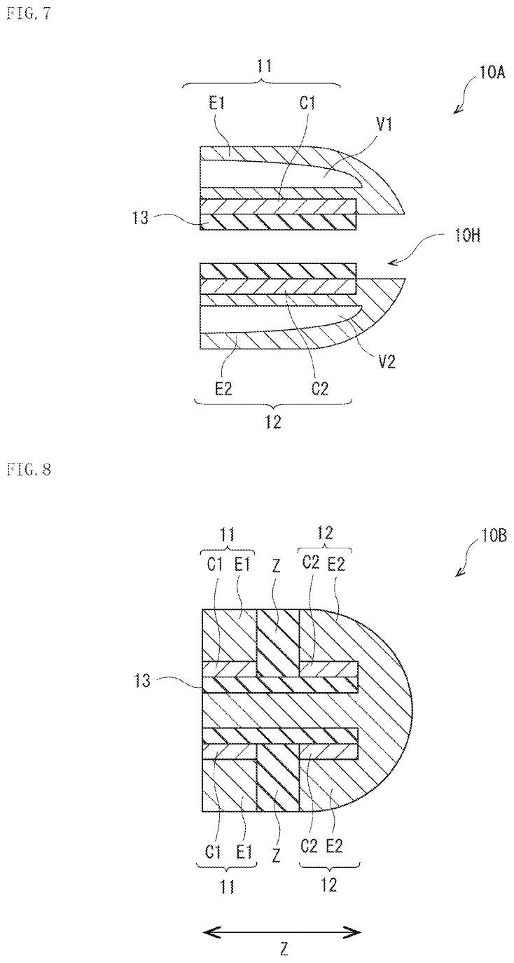

FIG. 5 is a schematic diagram illustrating a cross-sectional configuration example of the first particle 15. As illustrated in FIG. 5, the first particle 15 includes a base particle 151 and a surface treatment portion 152. The base particle 151 is an electric conductor or an electric insulator having a surface 151S, for example. Note that, although the base particle 151 having a spherical body is schematically illustrated in FIG. 5, the shape of the base particle is not limited to such a spherical body in the present disclosure. The base particle 151 may have an oval spherical shape or an irregular feature on the surface. The surface treatment portion 152 is, for example, an electric conductor that covers or substitutes at least a portion of the surface 151S of the base particle 151. Furthermore, the first particle 15 may not have the surface treatment portion 152 as long as the base particle 151 has electric conductivity. Furthermore, it is preferable that the surface treatment portion 152 be present over the entirety of the surface 151S of the base particle 151, as illustrated in FIG. 5. However, the first particles according to the present disclosure also include a first particle 15A having the surface treatment portion 152 present only on selective portions of the surface 151S of the base particle 151, as illustrated in FIG. 6, for example.

The base particles 151 may be carbon particles or metal particles, for example. One reason for this is that carbon particles and metal particles having electric conductivity themselves help to secure the electric conductivity of the first elastic portion E1L and the second elastic portion E2L. The metal particle serving as the base particle 151 includes at least one metal of gold (Au), silver (Ag), or copper (Cu), for example. When the base particle 151 is the metal particle, a metal compound serving as the surface treatment portion 152 may cover at least a portion of the surface 151S of the base particle 151. The metal compound serving as the surface treatment portion 152 is a sulfide, a selenide, or a chloride of at least one metal of gold (Au), silver (Ag), or copper (Cu), for example.

The carbon particle serving as the base particle 151 is a particle of a carbon compound of various kinds, for example. Specifically, the carbon particle is a particle of carbon black (graphite), carbon nanotubes, or graphene, for example. Alternatively, the carbon particles may be granules of flakes or fibers of a substance such as carbon black (graphite), carbon nanotubes, or graphene.

The base particle 151 may be a polymer particle, for example. Such a polymer particle is a particle of a resin composition of various kinds. Examples of the polymer particle may include particles of a polyphenylene sulfide (PPS) resin, a polyethylene terephthalate (PET) resin, a polyether sulfone (PES) resin, a polyamide-imide (PAI) resin, an acrylic resin, a polyvinylidene difluoride (PVDF) resin, an epoxy resin, a polylactic resin, a nylon resin, and a urethane resin.

When the base particle 151 is an electrically insulating particle, the first particle 15 may include an electrically conductive organic polymer as the surface treatment portion 152, for example. The electrically conductive organic polymer serving as the surface treatment portion 152 includes at least one selected from a group consisting of PEDOT-PSS, polypyrrole, polyacetylene, polyphenylenevinylene, polythiophene, polythiol, polyaniline, and analogs thereof. For the surface treatment portion 152, any of the electrically conductive organic polymers described above may be solely used, or some of the electrically conductive organic polymers described above may be used in combination. When the electrically conductive organic polymer is used as the surface treatment portion 152, the electrically conductive organic polymer may be mixed with a binder resin of various kinds, such as a water-soluble acrylic resin, a water-soluble urethane resin, water-soluble polyester alkyd, or a water-soluble amino resin. One reason for this is that the surface 151S of the base particle 151 is covered more securely, improving close adhesion between the base particle 151 and the surface treatment portion 152. Note that the electrically conductive organic polymer serving as the surface treatment portion 152 may be used also when the base particle 151 is an electrically conductive particle. Furthermore, the surface treatment portion 152 of the first particle 15 may have a portion that includes the electrically conductive organic polymer and a portion that includes the metal compound in a mixed manner.

### (First Electrically Insulating Portion Z1L and Second Electrically Insulating Portion Z2L)

As illustrated in FIG. 4, the first electrically insulating portion Z1L and the second electrically insulating portion Z2L each include a second elastic resin 16 and a plurality of second particles 17 buried in the second elastic resin 16. The second elastic resin 16 is an insulating resin, for example. A constituent material of the second elastic resin 16 may be substantially identical to the constituent material of the first elastic resin 14. The second particles 17 are particles of electric insulators, for example. For example, a titanium oxide such as TiO2 may be used as the second particles 17. The first electrically insulating portion Z1L and the second electrically insulating portion Z2L each include the second particles 17 at 20 wt %, for example.

The first elastic resin 14 and the second elastic resin 16 are resins each including a predetermined resin material, a predetermined rubber material, or a predetermined elastomer material as a main component. In more detail, the first elastic resin 14 and the second elastic resin 16 are each a solidified material or a hardened material of a resin component that includes a predetermined resin material, a predetermined rubber material, or a predetermined elastomer material as a main component. Herein, a resin component itself solidified is simply referred to as the “solidified material”, and a resin component mixed with a hardening agent of various kinds and thus hardened is referred to as the “hardened material”. Furthermore, the term “main component” represents a component included at 50 parts by mass or greater in a whole resin component of 100 parts by mass. The term “resin component” does not include non-resin components such as a crosslinking agent.

It is preferable that the first elastic resin 14 and the second elastic resin 16 each have a Shore hardness (Shore A hardness) of 70 HS or lower, for example. One reason for this is that, when the first elastic resin 14 has a Shore A hardness of 70 HS or lower, it is possible to allow the first electrically conductive portion 11L and the second electrically conductive portion 12L, formed by kneading the first particles 15 into the first elastic resin 14, to have a Shore A hardness of 80 HS or lower. Similarly, another reason of this is that, when the second elastic resin 16 has a Shore A hardness of 70 HS or lower, it is possible to allow the first electrically insulating portion Z1L and the second electrically insulating portion Z2L, formed by kneading the second particles 17 into the second elastic resin 16, to have a Shore A hardness of 80 HS or lower. By allowing the first electrically conductive portion 11L and the second electrically conductive portion 12L to each have a Shore A hardness of 80 HS or lower, it is possible to easily follow a movement of a biological body when the biological body moves, making it possible to further improve close adhesion with the biological body. Furthermore, comfortability is secured when the electrode body 10L is attached to the human body or the biological body. Note that the Shore A hardness of each of the first elastic resin 14 and the second elastic resin 16 is measured in accordance with a method described in JIS K6253:2012.

In the electrode body 10L, at least the first electrically conductive portion 11L having the first surface 11SL and the second electrically conductive portion 12L having the second surface 12SL that are to come into contact with a biological body include the first elastic resin 14 described above. It is therefore possible to sufficiently keep close adhesion between the electrode body 10L and the biological body.

Examples of the first elastic resin 14 and the second elastic resin 16 may include at least one thermoplastic resin selected from a group consisting of a polyvinylchloride resin, a polypropylene resin, a polyethylene resin, a polyurethane resin, a polyacetal resin, a polyamide resin, and a polycarbonate resin, and copolymers thereof.

Furthermore, the rubber material applied to the first elastic resin 14 and the second elastic resin 16 is natural rubber or synthetic rubber, for example. Examples of such synthetic rubber may include diene-based rubber, such as styrene butadiene rubber and isoprene rubber.

Furthermore, examples of the elastomer material applied to the first elastic resin 14 and the second elastic resin 16 include a thermosetting elastomer including one selected from a group consisting of a silicone resin and a polyurethane resin.

Note that the resin materials, the rubbers, and the elastomers described above are mere examples. In addition to those described above, various kinds of known resin materials, known rubbers, and known elastomers may be used. Furthermore, the first elastic resin 14 and the second elastic resin 16 may include various additives such as pigments and coloring agents.

As illustrated in FIG. 4, the through hole 10HL is provided to receive the support member 13L therein. The support member 13L is a member that has a shape allowing it to be press-fitted into the through hole 10HL. For example, the support member 13L has a substantially cylindrical shape or a substantially columnar shape. The support member 13L includes the first electrode 131, the second electrode 132, and insulating portions 133. The first electrode 131 is a portion in contact with the first core portion C1L. The second electrode 132 is a portion in contact with the second core portion C2L. The insulating portion 133 is an electric insulator that electrically separates the first electrode 131 and the second electrode 132 from each other.

### (Electrode Body 10R)

A configuration of the electrode body 10R is substantially identical to the configuration of the electrode body 10L described above. Specifically, for example, the configuration of the electrode body 10R may be described by replacing the first electrically conductive portion 11L, the second electrically conductive portion 12L, the first electrically insulating portion Z1L, the second electrically insulating portion Z2L, the through hole 10HL, the interface K1L, the interface K2L, the interface K3L, the interface K4L, the first core portion C1L, the first elastic portion E1L, the first surface 11SL, the second core portion C2L, the second elastic portion E2L, and the second surface 12SL of the electrode body 10L with a first electrically conductive portion 11R, a second electrically conductive portion 12R, a first electrically insulating portion Z1R, a second electrically insulating portion Z2R, a through hole 10HR, an interface KlR, an interface K2R, an interface K3R, an interface K4R, a first core portion ClR, a first elastic portion E1R, a first surface 11SR, a second core portion C2R, a second elastic portion E2R, and a second surface 12SR that are illustrated in FIG. 4. As illustrated in FIG. 4, a support member 13R is provided so as to be insertable into the through hole 10HR. A configuration of the support member 13R is substantially identical to the configuration of the support member 13L.

Note that the first electrically conductive portion 11L and the first electrically conductive portion 11R may be hereinafter collectively referred to as the first electrically conductive portion 11. Similarly, the second electrically conductive portion 12L and the second electrically conductive portion 12R may be collectively referred to as the second electrically conductive portion 12. Further, the first electrically insulating portion Z1L and the first electrically insulating portion Z1R may be collectively referred to as the first electrically insulating portion Z1. Further, the second electrically insulating portion Z2L and the second electrically insulating portion Z2R may be collectively referred to as the second electrically insulating portion Z2. Further, the through hole 10HL and the through hole 10HR may be collectively referred to as the through hole 10H. Further, the interface K1L, the interface K2L, the interface K3L, the interface K4L, the interface K1R, the interface K2R, the interface K3R, and the interface K4R may be collectively referred to as the interface K. Further, the first elastic portion E1L and the first elastic portion E1R may be collectively referred to as the first elastic portion E1. The first surface 11SL and the first surface 11SR may be collectively referred to as the first surface 11S. Further, the second elastic portion E2L and the second elastic portion E2R may be collectively referred to as the second elastic portion E2. Further, the second surface 12SL and the second surface 12SR may be collectively referred to as the second surface 12S.

### 1.2. Manufacturing Method

### (Method of Manufacturing Electrode Body 10)

Next, a method of manufacturing the electrode body 10 according to the present embodiment will be briefly described. The electrode body 10 may be manufactured using a so-called injection molding technology. The manufacturing process includes first to third steps, for example. The first step is a step of manufacturing the first particles 15 and the second particles 17. The second step is a step of manufacturing a first kneaded resin composition and a second kneaded resin composition by kneading the manufactured first particles 15 and the manufactured second particles 17 with the first elastic resin 14 and the second elastic resin 16, respectively. The third step is a step of manufacturing the electrode body 10 in which the first electrically conductive portion 11 and the second electrically conductive portion 12 are alternately disposed and integrated with each other through simultaneous injection molding of the first kneaded resin composition and the second kneaded resin.

Here, the method of manufacturing the first particles 15 and the second particles 17 is not particularly limited. For the first particles 15, a known method may be employed, such as one that allows a chemical compound for forming the surface treatment portions 152 to permeate in a vessel filled with the base particles 151, or one that adds the base particles 151 into a solution including a chemical compound for forming the surface treatment portions 152 and agitates the solution, for example. The methods of manufacturing the first kneaded resin composition and the second kneaded resin composition are also not particularly limited, and a known method may be employed to manufacture these resin compositions. Note that an average particle diameter of a secondary aggregate (agglomerate) of the first particle 15 to be kneaded into the first elastic resin 14 ranges, for example, from approximately 1 μm to 200 μm both inclusive.

Furthermore, as to the third step described above, a known injection molding technology of various kinds, such as a bi-color molding technique, may be used to manufacture the electrode body 10 having a desired size and a desired shape.

### 1.3. Workings and Effects

According to the present embodiment, the electrode body 10 has the first electrically conductive portion 11, the second electrically conductive portion 12, the first electrically insulating portion Z1, and the second electrically insulating portion Z2. The first electrically conductive portion 11 and the second electrically conductive portion 12 have the first surface 11S and the second surface 12S, respectively. The first surface 11S and the second surface 12S serve as contact surfaces contactable with the biological body. Note that the first electrically conductive portion 11 and the second electrically conductive portion 12 are electrically insulated from each other by the first electrically insulating portion Z1 and the second electrically insulating portion Z2. It is therefore possible to detect the electric potential of the biological body at each of the contact point between the first surface 11S of the first electrically conductive portion 11 and the biological body and the contact point between the second surface 12S of the second electrically conductive portion 12 and the biological body. That is, the electrode body 10 may be used to detect electric potential at a plurality of locations in the biological body.

Furthermore, in the electrode body 10 according to the present embodiment, the first electrically conductive portion 11 has the first elastic portion E1, and the second electrically conductive portion 12 has the second elastic portion E2. It is therefore possible to appropriately keep a close-adhesion state between the first surface 11S and the biological body and a close-adhesion state between the second surface 12S and the biological body.

Furthermore, in the electrode body 10, the first elastic portion E1 and the second elastic portion E2 each include the first elastic resin 14 and the plurality of first particles 15. It is therefore possible to form the first elastic resin 14 with an insulating resin by using the first particles 15 as electric conductors, for example. It is therefore possible to improve ease of processing, as compared with a case where the first elastic resin 14 is formed with a conductive resin. This makes it possible to form at higher accuracy the first elastic portion E1 and the second elastic portion E2 having further various shapes.

Furthermore, in the electrode body 10, the first electrically insulating portion Z1, and the second electrically insulating portion Z2 each include the second elastic resin 16 and the plurality of second particles 17. Thus, as compared with a case where the first electrically insulating portion Z1 and the second electrically insulating portion Z2 each do not include the second particles 17, peeling off is less likely to occur at the interface K between the first electrically conductive portion 11 and the second electrically conductive portion 12 and the interface K between the first electrically insulating portion Z1 and the second electrically insulating portion Z2. One reason for this is that, since the first electrically conductive portion 11 and the second electrically conductive portion 12 each include the first particles 15, and the first electrically insulating portion Z1 and the second electrically insulating portion Z2 each include the second particles 17, stress concentration is mitigated at the interfaces K. The electrode body 10 is therefore improved in mechanical strength, and is thus improved in long-term reliability.

In particular, since the first elastic resin 14 and the second elastic resin 16 include a substantially identical constituent material, stress concentration is further mitigated at the interfaces K. As a result, the electrode body 10 is further improved in mechanical strength, and is thus further improved in long-term reliability.

Furthermore, in the electrode body 10, the first electrically conductive portion 11, the first electrically insulating portion Z1, the second electrically conductive portion 12, and the second electrically insulating portion Z2 are alternately disposed in the circumferential direction of the cylindrical structure. It is therefore possible to allow each of the first electrically conductive portion 11 and the second electrically conductive portion 12 to come into contact with respective measuring portions of the biological body. When the electrode body 10 is attached to an external acoustic pore of the human body to detect brain waves (an electroencephalogram: EEG) of the human body, the first surface 11S of the first electrically conductive portion 11 may be allowed to come into contact with a portion near the top of the head, where EEG strength is relatively higher, on an inner surface of the external acoustic pore, and the second surface 12S of the second electrically conductive portion 12 may be allowed to come into contact with a portion near the neck, where the EEG strength is relatively lower, on the inner surface of the external acoustic pore, for example. In that case, it is possible to measure an electric potential difference between the electric potential at the portion near the top of the head and the electric potential at the portion near the neck.

### 1.4. Modification Examples

### Modification Example 1-1

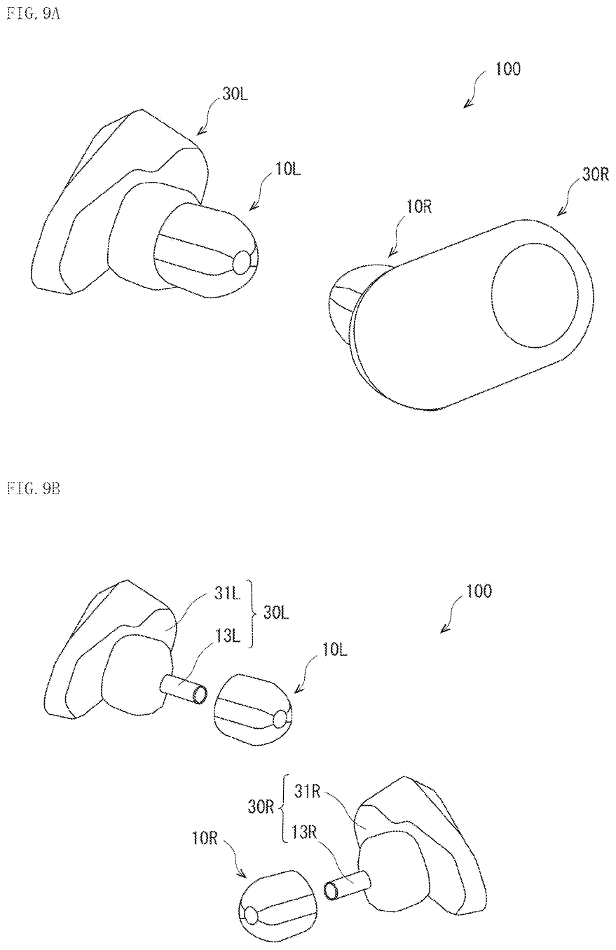

FIG. 7 is a cross-sectional view illustrating a configuration example of an electrode body 10A according to a first modification example to the first embodiment of the present disclosure (Modification Example 1-1). In the first embodiment described above, the first elastic portion E1 and the second elastic portion E2 of the electrode body 10 are solid bodies including no cavities and no air bubbles. Alternatively, the electrode body 10A according to Modification Example 1-1 has a cavity V1 and a cavity V2 inside the first elastic portion E1 and the second elastic portion E2, respectively. With the electrode body 10A according to Modification Example 1-1, which has the cavities V1, V2, the first electrically conductive portion 11 and the second electrically conductive portion 12 have further higher elasticity. It is therefore possible to keep a close adhesion state more appropriately between the first surface 11S and the biological body and a close-adhesion state between the second surface 12S and the biological body. Note that respective cavities may be further provided inside the first electrically insulating portion Z1 and the second electrically insulating portion Z2.

### Modification Example 1-2

FIG. 8 is a cross-sectional view illustrating a configuration example of an electrode body 10B according to a second modification example to the first embodiment of the present disclosure (Modification Example 1-2). The electrode body 10 according to the first embodiment described above has a cylindrical structure. In the electrode body 10, the electrically conductive portions and the electrically insulating portions extend from the center of the cylindrical structure in respective radial directions. However, in the electrode body 10B according to Modification Example 1-2, multiple electrically conductive portions are separated from each other by electrically insulating portions in an axial direction of the cylindrical structure. That is, in the electrode body 10B according to Modification Example 1-2, the first electrically conductive portion 11, an electrically insulating portion Z, and the second electrically conductive portion 12 are sequentially provided in this order in a Z-axis direction indicated by an arrow. Here, the electrically insulating portion Z has a configuration identical to that of at least one of the first electrically insulating portion Z1 or the second electrically insulating portion Z2, for example. In the electrode body 10B according to Modification Example 1-2, the first electrically conductive portion 11 and the second electrically conductive portion 12 are each provided over an entire circumference without being interrupted in the circumferential direction of the cylindrical structure. This eliminates the need for alignment between a position of the cylindrical structure in the circumferential direction and a position of the inner surface of the external acoustic pore when the electrode body 10B is attached to an external acoustic pore of the human body, for example. Furthermore, as will be described later, this configuration is advantageous when the first electrically conductive portion 11 is used as a terminal for biopotential detection and the second electrically conductive portion 12 is used as a reference terminal or a grounding terminal.

### 2. Second Embodiment

### 2.1. Configuration Example

### (Biopotential Sensor 100)

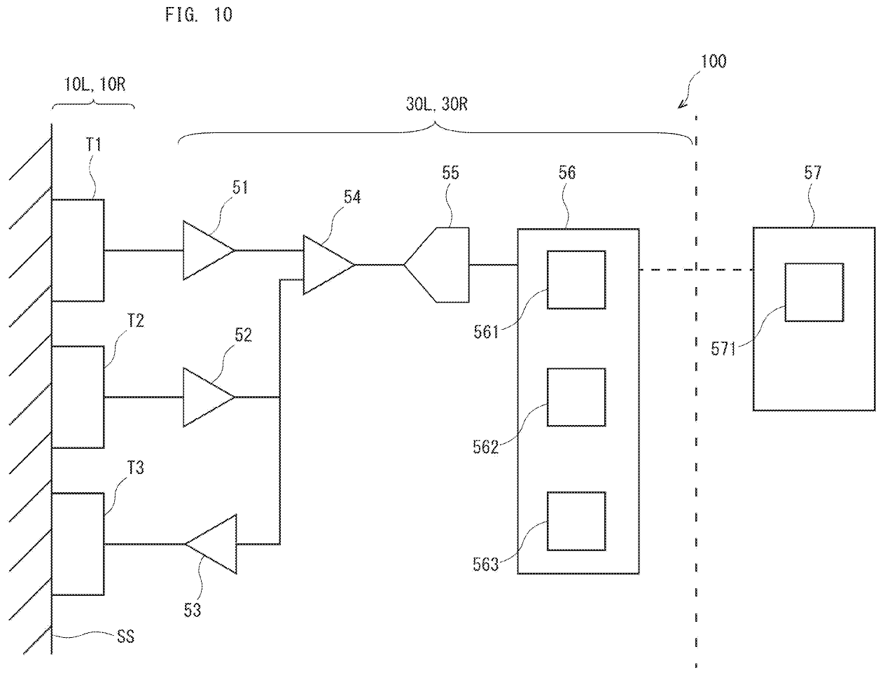

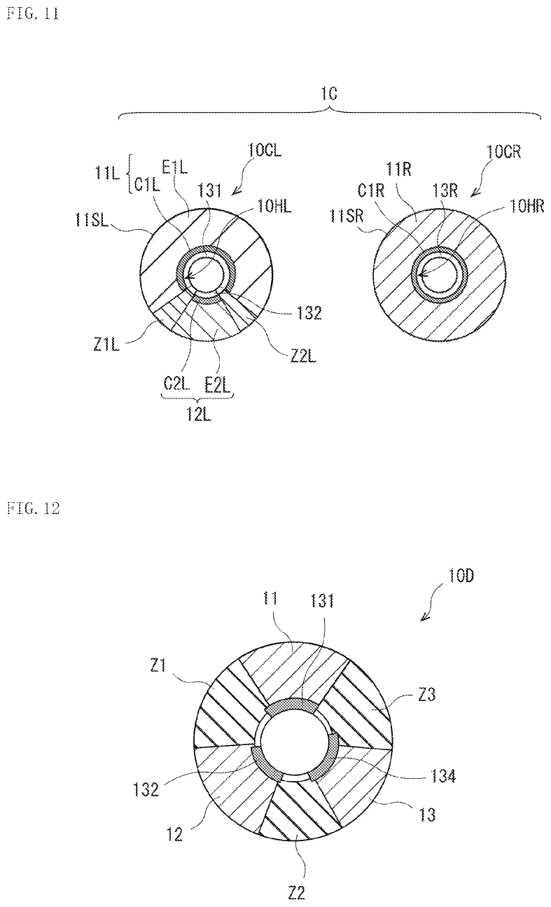

Next, a biopotential sensor 100 according to a second embodiment of the present disclosure will be described with reference to FIGS. 9A to 10. FIG. 9A is a perspective view schematically illustrating an overall configuration example of the biopotential sensor 100. FIG. 9B is an exploded perspective view of the biopotential sensor 100. FIG. 10 is a block diagram schematically illustrating the overall configuration example of the biopotential sensor 100.

The biopotential sensor 100 is mounted in wireless earphones. As illustrated in FIG. 9A, the biopotential sensor 100 includes the electrode bodies 10L and 10R and main bodies 30L and 30R. FIG. 9B illustrates a state where the electrode bodies 10L and 10R are removed from the respective main bodies 30L and 30R. As illustrated in FIG. 9B, the main body 30L includes a base 31L and the support member 13L provided to the base 31L. The main body 30R includes a base 31R and the support member 13R provided to the base 31R. The electrode bodies 10L and 10R each have the configuration according to the first embodiment described above. The electrode bodies 10L and 10R are supported by the respective support members 13L and 13R inserted into the respective through holes 0HL and 10HR.

As illustrated in FIG. 10, the biopotential sensor 100 includes terminals T1 to T3, pre-amplifiers 51 to 53, a differential amplifier 54, an analog-digital converter (ADC) 55, and a signal processing device 56, for example. The terminals T1 to T3 are included in the electrode bodies 10L and 10R. The pre-amplifiers 51 to 53, the differential amplifier 54, the ADC 55, and the signal processing device 56 are included in the main bodies 30L and 30R. The signal processing device 56 includes a signal processing unit 561, a memory unit 562, and a communication unit 563, for example. The signal processing unit 561 includes a digital signal processor (DSP), for example. The memory unit 562 includes a semiconductor memory such as a dynamic random access memory (DRAM). The communication unit 563 includes a communication device that conforms to a short-range wireless communications standard including Bluetooth (registered trademark of Bluetooth SIG, Inc.), for example. The signal processing devices 56 may be communicably coupled to an external device 57 such as a personal computer (PC) in a wireless or wired manner, for example. The external device 57 includes an arithmetic operation unit 571 such as a central arithmetic processing device (CPU), for example.

The terminals T1 to T3 are allowed to come into contact with a skin surface SS of the biological body at respective locations different from each other. The biopotential sensor 100 may be used to measure brain waves of the human body by attaching the electrode body 10L to the left external acoustic pore of the human body and the electrode body 10R to the right external acoustic pore of the human body, for example. In that case, the first electrically conductive portion 11L of the electrode body 10L and the first electrically conductive portion 11R of the electrode body 10R of the biopotential sensor 100 are used as the terminal T1, for example. The first surface 11SL of the first electrically conductive portion 11L and the first surface 11SR of the first electrically conductive portion 11R may be allowed to come into contact with respective portions near the top of the head, where the EEG strength is relatively higher, on the inner surfaces of the left and right external acoustic pores. Furthermore, the second electrically conductive portion 12L of the electrode body 10L is used as the terminal T2, for example. In that case, the second surface 12SL of the second electrically conductive portion 12L may be allowed to come into contact with a portion near the neck, where the EEG strength is relatively lower, on the inner surface of the external acoustic pore. The terminal T2 is, for example, a reference terminal that detects reference electric potential to measure a difference from the electric potential detected by the terminal T1, for example. Further, the second electrically conductive portion 12R of the electrode body 10R is used as the terminal T3, for example. In that case, the second surface 12SR of the second electrically conductive portion 12R may be allowed to come into contact with a portion near the neck, where the EEG strength is relatively lower, on the inner surface of the external acoustic pore. The terminal T3 is, for example, a grounding terminal.

### 2.2. Workings and Effects

As described above, the biopotential sensor 100 according to the present embodiment, which includes the electrode bodies 10L and 10R according to the first embodiment described above, may be used to detect electric potential at a plurality of locations in the biological body. For example, the first surface 11S of the first electrically conductive portion 11 is allowed to come into contact with a portion near the top of the head, where the EEG strength is relatively higher, on the inner surface of the external acoustic pore, and the second surface 12S of the second electrically conductive portion 12 is allowed to come into contact with a portion near the neck, where the EEG strength is relatively lower, on the inner surface of the external acoustic pore. This makes it possible to easily measure an electric potential difference between the electric potential at the portion near the top of the head and the electric potential at the portion near the neck. It is therefore possible to accurately detect brain waves of the human body. Furthermore, the electrode bodies 10 having superior mechanical strength improve the biopotential sensor 100 in long-term reliability.

### 2.3. Modification Examples

### Modification Example 2-1

FIG. 11 is a schematic diagram illustrating a cross-sectional configuration example of an electrode body unit 1C of a biopotential sensor 100A according to a first modification example to the second embodiment of the present disclosure (Modification Example 2-1). The biopotential sensor 100 according to the second embodiment described above uses the electrode body 10L and the electrode body 10R having configurations substantially identical to each other. In contrast, the electrode body unit 1C according to Modification Example 2-1 includes an electrode body 10CL and an electrode body 10CR having configurations different from each other. Specifically, as illustrated in FIG. 11, the electrode body 10CR to be attached to the right external acoustic pore is provided with the first electrically conductive portion 11R that occupies the entire area in the circumferential direction of the cylindrical structure, for example. In contrast, the electrode body 10CL to be attached to the left external acoustic pore is provided with the first electrically conductive portion 11L, the first electrically insulating portion Z1L, the second electrically conductive portion 12L, and the second electrically insulating portion Z2L that are alternately disposed in the circumferential direction of the cylindrical structure. Note that, in the electrode body 10CL, a volume ratio at which the first electrically conductive portion 11L occupies in the entire circumference of the electrode body 10L is greater than a volume ratio at which the second electrically conductive portion 12L occupies in the entire circumference of the electrode body 10L. Note that the support member 13L is provided with the first electrode 131 corresponding to the first electrically conductive portion 11L, and the second electrode 132 corresponding to the second electrically conductive portion 12L. The support member 13R is provided with the first electrode 131 corresponding to the first electrically conductive portion 11R.

In the electrode body unit 1C of the biopotential sensor 100A according to Modification Example 2-1, the first electrically conductive portion 11R of the electrode body 10CR may be used as the terminal T1 for detecting brain waves, the first electrically conductive portion 11L of the electrode body 10CL may be used as the terminal T2 for reference, and the second electrically conductive portion 12L of the electrode body 10CL may be used as the terminal T3 for grounding, for example.

### Modification Example 2-2

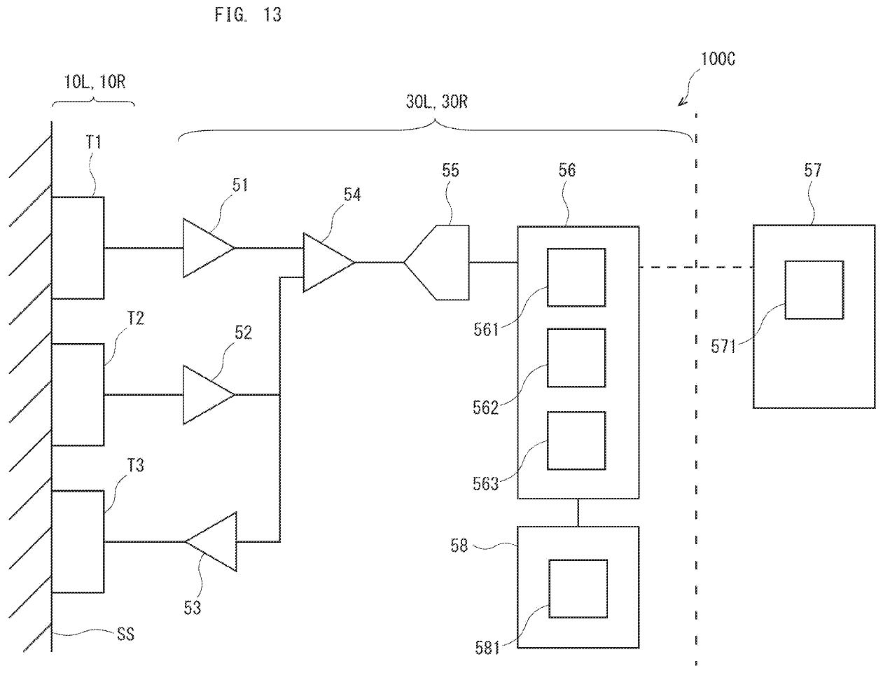

FIG. 12 is a schematic diagram illustrating a cross-sectional configuration example of an electrode body 10D of a biopotential sensor 100B according to a second modification example to the second embodiment of the present disclosure (Modification Example 2-2). In the second embodiment described above, the electrode body unit 1 including the pair of electrode bodies 10L and 10R is used. In contrast, in the biopotential sensor 100B according to Modification Example 2-2, the terminals T1 to T3 are assigned to the single electrode body 10D. When the biopotential sensor 100B is used to detect brain waves of the human body, for example, the electrode body 10D is attached to the right or left external acoustic pore. As illustrated in FIG. 12, the electrode body 10D includes three electrically conductive portions, i.e., first to third electrically conductive portions 11 to 13. The electrode body 10D further includes first to third electrically insulating portions Z1 to Z3 that are provided to electrically separate the first to third electrically conductive portions 11 to 13 from each other. Note that the support member 13 is provided with the first electrode 131 corresponding to the first electrically conductive portion 11, the second electrode 132 corresponding to the second electrically conductive portion 12, and a third electrode 134 corresponding to a third electrically conductive portion 13.

With the biopotential sensor 100B according to Modification Example 2-2, it is possible to easily detect biopotential by simply attaching the electrode body 10D to the biological body.

### Modification Example 2-3

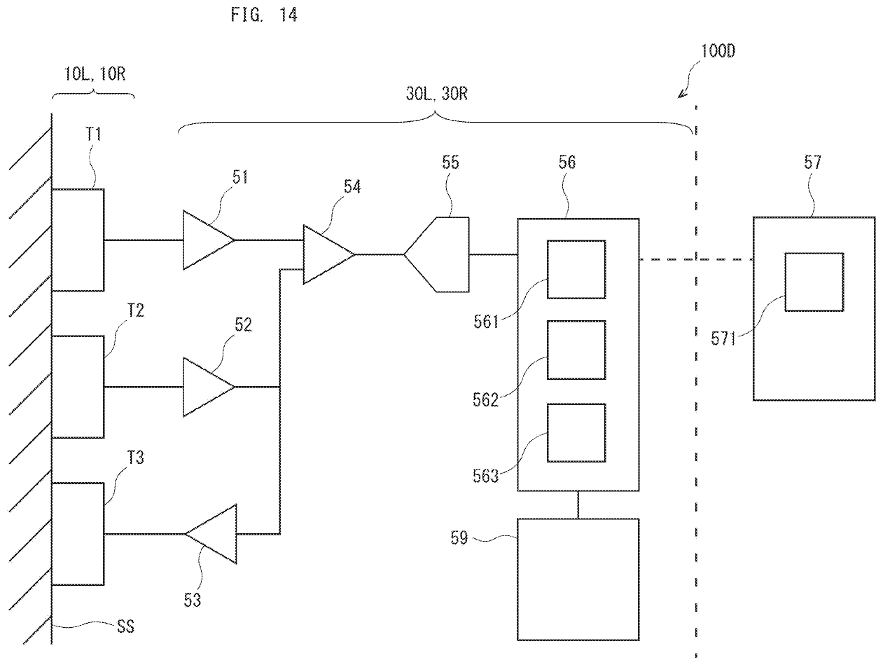

FIG. 13 illustrates a biopotential sensor 100C according to a third modification example to the second embodiment of the present disclosure (Modification Example 2-3). FIG. 13 is a block diagram schematically illustrating an overall configuration example of the biopotential sensor 100C.

In the biopotential sensor 100C according to the present embodiment, the signal processing unit 561 determines whether the electrode bodies 10L and 10R are being attached to the biological body on the basis of biological signals acquired from the biological body, for example. That is, the signal processing unit 561 serves as a determination unit to determine whether the electrode body 10L is being attached to the biological body on the basis of a change in impedance of the biological body between at the first electrically conductive portion 11L and at the second electrically conductive portion 12L in the electrode body 10L, for example. Similarly, the signal processing unit 561 serves as a determination unit to determine whether the electrode body 10R is being attached to the biological body on the basis of a change in impedance of the biological body between at the first electrically conductive portion 11R and at the second electrically conductive portion 12R in the electrode body 10R.

The signal processing unit 561 may determine whether the electrode bodies 10L and 10R are being attached to the biological body on the basis of resistance of the measure impedance values of the biological body, out of resistance and reactance of the measured impedance values, for example.

As illustrated in FIG. 13, the biopotential sensor 100C may further include a current supplying unit 58 including a secondary battery 581 and other components, for example. The current supplying unit 58 supplies a current between the first electrically conductive portion 11L and the second electrically conductive portion 12L, and between the first electrically conductive portion 11R and the second electrically conductive portion 12R. The current is in the form of a sine wave, a rectangular wave, or a triangular wave at a frequency of greater than 0 Hz and less than or equal to 1000 Hz, for example. Note that, although FIG. 13 illustrates an example where the current supplying unit 58 is provided inside each of the main bodies 30L and 30R, the current supplying unit 58 may be provided outside each of the main bodies 30L and 30R.

With the biopotential sensor 100C according to Modification Example 2-3, it is possible to easily detect at high accuracy whether the electrode bodies 10L and 10R are being attached to respective portions of the biological body.

Note that Japanese Unexamined Patent Application Publication (Published Japanese Translation of PCT Application) No. 2018-515045, for example, proposes a method of detecting, with a sensor such as an optical proximity sensor, a pressure sensor, a thermal sensor, or a moisture sensor, whether wireless earphones are being attached to the ears of a user. However, since an ear shape significantly varies depending on users, there may be cases where a determination as to whether a device such as wireless earphones is in an attached state is difficult depending on a user. For example, when an optical proximity sensor is used to determine whether an attached state is attained or not, that wireless earphones may be erroneously determined as being attached to the ears, even though the wireless earphones are actually placed on a table.

In contrast, in the biopotential sensor 100C according to Modification Example 2-3, the signal processing unit 561 serving as the determination unit determines whether the electrode bodies 10L and 10R are being attached to the biological body on the basis of biological signals such as bioelectrical impedance acquired from the biological body. It is therefore possible to eliminate a possibility of erroneously determining the state where the device is placed on a table as the state where the device is being attached to an ear, for example. Furthermore, the electrode bodies 10L and 10R each include the first electrically conductive portion 11 including the first elastic portion E1, and the second electrically conductive portion 12 including the second elastic portion E2. It is therefore possible to appropriately keep a close-adhesion state between the electrode bodies 10L and 10R and the biological body. To acquire the bioelectrical impedance, measurement of impedance is performed between two points, in general. Using the electrode bodies 10L and 10R, it is possible to achieve higher adhesion even in a minute section between such two points in one acoustic pore. It is therefore possible for the biopotential sensor 100C to detect at high accuracy that the electrode bodies 10L and 10R are being attached to the biological body.

### Modification Example 2-4

For the biopotential sensor 100C according to Modification Example 2-3 described above, the bioelectrical impedance is detected to determine whether the electrode bodies 10L and 10R are being attached to the biological body. However, the present disclosure is not limited to this configuration. FIG. 14 is a block diagram schematically illustrating an overall configuration example of a biopotential sensor 100D according to a fourth modification example to the second embodiment of the present disclosure (Modification Example 2-4).

The biopotential sensor 100D according to Modification Example 2-4 further includes an oscillation circuit 59 that causes an electrical oscillation, in addition to the components of the biopotential sensor 100 according to the embodiment described above. In the biopotential sensor 100D, the signal processing unit 561 serves as a determination unit to determine whether the electrode bodies 10L and 10R are being attached to the biological body on the basis of a change in oscillating frequency of the electrical oscillation caused by the oscillation circuit 59. That is, the signal processing unit 561 detects a change in electric charge amount in each of the electrode bodies 10L and 10R on the basis of a change in frequency in the oscillation circuit 59 to determine whether the electrode bodies 10L and 10R are being in contact with the respective acoustic pores of the biological body.

With the biopotential sensor 100D according to Modification Example 2-4, it is also possible to easily detect at high accuracy whether the electrode bodies 10L and 10R are being attached to respective portions of the biological body.

### 3. Other Modification Examples

The technology according to the present disclosure has been described above with reference to some embodiments and modification examples. However, the technology according to the present disclosure is not limited to the embodiments and the modification examples described above, and various modification may be made.

In the first embodiment, for example, the first elastic portion E1 and the second elastic portion E2 of the electrode body 10 are solid bodies including no cavities and no air bubbles. However, the present disclosure is not limited to this case. For example, the first elastic resin used for the electrically conductive portions may be a foam body including air bubbles. Similarly, the second elastic resin used for the electrically insulating portions may be a solid body including no cavity and no air bubbles, or a foam body including air bubbles.

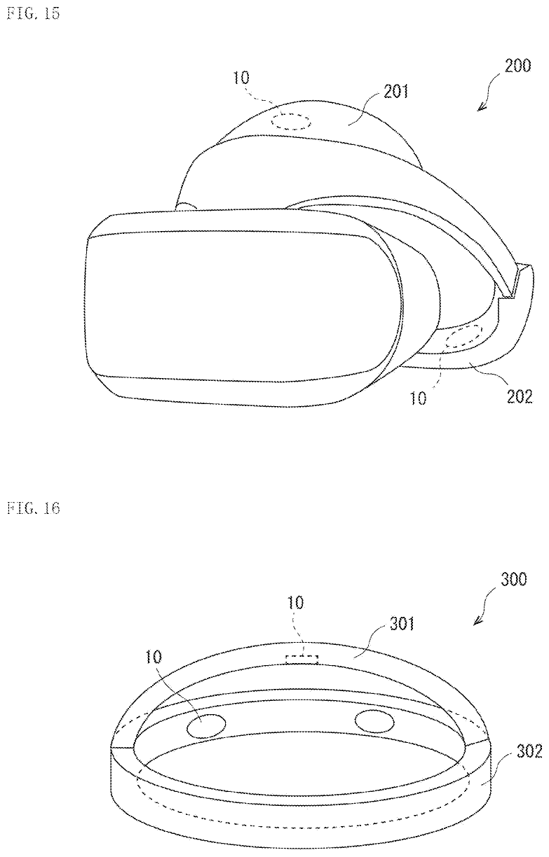

Furthermore, in the second embodiment, for example, the wireless earphone-type biopotential sensor 100 or the like is used as a biopotential sensor. However, the present disclosure is not limited to the embodiment. A biopotential sensor according to the present disclosure may be provided as a portion of a head mount display 200 illustrated in FIG. 15, for example. For the head mount display 200, the electrode bodies 10 may be provided on inner surfaces of a pad part 201 and a band part 202, for example.

Alternatively, the biopotential sensor according to the present disclosure may be provided as a portion of a headband 300 illustrated in FIG. 16, for example. For the headband 300, the electrode bodies 10 may be provided on inner surfaces of band parts 301 and 302, which come into contact with the head, for example.

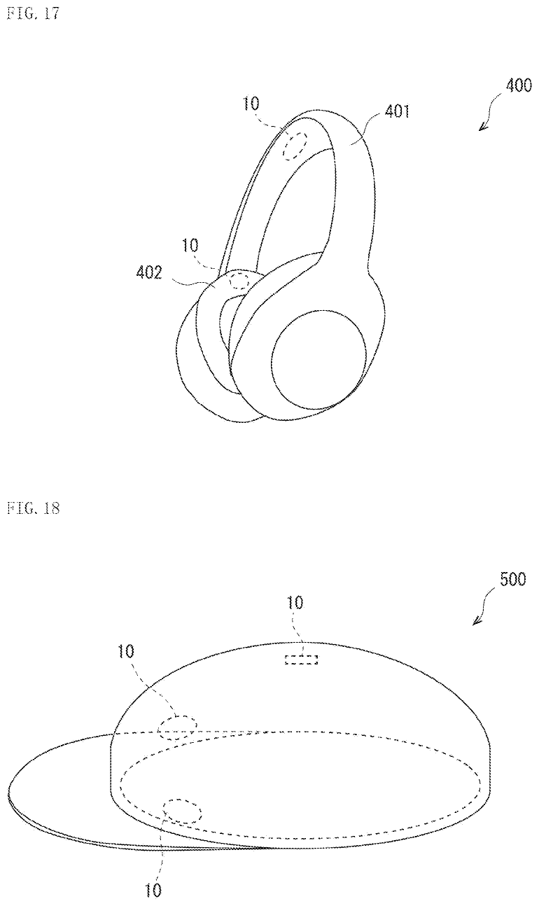

Alternatively, the biopotential sensor according to the present disclosure may be provided as a portion of a headphone 400 illustrated in FIG. 17, for example. For the headphone 400, the electrode bodies 10 may be provided on an inner surface of a band part 401 or earpads 402, which come into contact with the head, for example.

Alternatively, the biopotential sensor according to the present disclosure may be provided as a portion of a cap 500 illustrated in FIG. 18, for example. For the cap 500, the electrode bodies 10 may be provided on respective portions of an inner surface, which come into contact with the forehead, for example.

Furthermore, all the configurations and actions described in the embodiments are not necessarily essential configurations and actions of the present disclosure. For example, components in the embodiments which are not recited in a most-generic independent claim of the disclosure may be regarded as optional components.

It should be construed that the terms used in the whole of the present specification and claims are “non-limiting” terms. For example, it should be construed that the terms “include” or “included” are “not limited to those configurations that are described to be included”. It should be construed that the term “have” is “not limited to those configurations that are described to have”.

The terms used in the present specification are used merely for purpose of description, and include terms that are not used for purpose of limiting the configurations and actions. For example, the terms “right”, “left”, “upper”, and “lower” merely indicate directions in the drawings when referred to. Furthermore, the terms “inside” and “outside” merely indicate a direction toward the center of an element of interest, and a direction away from the center of the element of interest, respectively. The same applies to other terms similar to those described above and other terms with the same meaning.

Note that the technology according to the present disclosure may have such configurations as those described below. The biopotential electrode body according to the present disclosure with the following configurations has high mechanical strength and is suitable for acquiring more bioinformation. Note that the effects of the technology according to the present disclosure are not necessarily limited to those effects described above, and may be any effects described in the present disclosure.

(1) A biopotential electrode body includes:
multiple electrically conductive portions each having a contact surface contactable with a biological body; and
one or more electrically insulating portions that electrically insulate the multiple electrically conductive portions from each other, in which
the electrically conductive portions each include a first elastic resin and a plurality of first particles buried in the first elastic resin, and
the electrically insulating portions each include a second elastic resin and a plurality of second particles buried in the second elastic resin.

(2) The biopotential electrode body according to (1) described above, in which
the first particles are electric conductors, and
the second particles are electric insulators.

(3) The biopotential electrode body according to (2) described above, in which the first elastic resin and the second elastic resin are electric insulators.

(4) The biopotential electrode body according to (3) described above, in which the first elastic resin and the second elastic resin are materials substantially identical to each other.

(5) The biopotential electrode body according to any one of (1) to (4) described above, in which the first particles include carbon particles or metal particles.

(6) The biopotential electrode body according to (5) described above, in which the metal particles each include at least one of gold (Au), silver (Ag), or copper (Cu).

(7) The biopotential electrode body according to any one of (1) to (6) described above, in which the first particles each include metal particles and a metal compound, the metal compound covering at least a portion of a surface of each of the metal particles.

(8) The biopotential electrode body according to (7) described above, in which the metal compound is a sulfide, a selenide, or a chloride of at least one metal of gold (Au), silver (Ag), or copper (Cu).

(9) The biopotential electrode body according to any one of (1) to (8) described above, in which the first particles each include a base particle and an electrically conductive organic polymer covering at least a portion of a surface of the base particle.

(10) The biopotential electrode body according to (9) described above, in which the electrically conductive organic polymer includes at least one selected from a group consisting of PEDOT-PSS, polypyrrole, polyacetylene, polyphenylenevinylene, polythiophene, polythiol, polyaniline, and analogs thereof.

(11) The biopotential electrode body according to any one of (1) to (10) described above, in which the first elastic resin and the second elastic resin are each at least one thermoplastic resin selected from a group consisting of a polyvinylchloride resin, a polypropylene resin, a polyethylene resin, a polyurethane resin, a polyacetal resin, a polyamide resin, a polycarbonate resin, and copolymers thereof.

(12) The biopotential electrode body according to any one of (1) to (10) described above, in which the first elastic resin and the second elastic resin are each natural rubber or diene rubber.

(13) The biopotential electrode body according to any one of (1) to (10) described above, in which the first elastic resin and the second elastic resin are each a thermosetting elastomer including one selected from a group consisting of a silicone resin and a polyurethane resin.

(14) The biopotential electrode body according to any one of (1) to (13) described above, in which the second particles each include a titanium oxide.

(15) The biopotential electrode body according to any one of (1) to (14) described above, in which
the multiple electrically conductive portions and the one or more electrically insulating portions form a cylindrical structure as a whole, the cylindrical structure having a substantially cylindrical shape, and
the electrically conductive portions and the electrically insulating portions are alternately disposed in a circumferential direction of the cylindrical structure.

(16) The biopotential electrode body according to any one of (1) to (14) described above, in which
the multiple electrically conductive portions and the one or more electrically insulating portions form a cylindrical structure as a whole, the cylindrical structure having a substantially cylindrical shape, and
the multiple electrically conductive portions are separated from each other by the one or more electrically insulating portions in an axial direction of the cylindrical structure.

(17) A biopotential sensor includes a biopotential electrode body, the biopotential electrode body including:
multiple electrically conductive portions each having a contact surface contactable with a biological body; and
one or more electrically insulating portions that electrically insulate the multiple electrically conductive portions from each other, in which
the electrically conductive portions each include a first elastic resin and a plurality of first particles buried in the first elastic resin,
the electrically insulating portions each include a second elastic resin and a plurality of second particles buried in the second elastic resin.

(18) The biopotential electrode body according to (17) described above, in which
the multiple electrically conductive portions include a first electrically conductive portion and a second electrically conductive portion,
the first electrically conductive portion is a measurement terminal that measures electric potential in the biological body, and
the second electrically conductive portion is a grounding terminal.

(19) The biopotential electrode body according to (17) or (18) described above, further including a differential amplifier circuit, in which
the multiple electrically conductive portions include a first electrically conductive portion and a second electrically conductive portion,
the first electrically conductive portion is a measurement terminal that measures electric potential in the biological body,
the second electrically conductive portion is a reference terminal that measures reference electric potential, and
the differential amplifier circuit detects a difference between the electric potential in the biological body and the reference electric potential.

(20) The biopotential sensor according to (17) described above, further including a determination unit that determines that the biopotential electrode body is being attached to the biological body on the basis of a biological signal acquired from the biological body.

(21) The biopotential sensor described in (20) above, in which
the multiple electrically conductive portions include a first electrically conductive portion and a second electrically conductive portion, and
the determination unit determines that the biopotential electrode body is being attached to the biological body on the basis of a change in impedance of the biological body between at the first electrically conductive portion and at the second electrically conductive portion.

(22) The biopotential sensor according to (21) described above, in which the determination unit determines that the biopotential electrode body is being attached to the biological body on the basis of resistance of a measured impedance value of the biological body.

(23) The biopotential sensor according to (21) or (22) described above, further including a current supplying unit that supplies a current having a frequency of greater than 0 Hz and less than or equal to 1000 Hz between the first electrically conductive portion and the second electrically conductive portion.

(24) The biopotential sensor according to (17) described above, further including:
an oscillation circuit that causes an electrical oscillation; and
a determination unit that determines that the biopotential electrode body is being attached to the biological body on the basis of a change in oscillating frequency of the electrical oscillation.

This application claims the benefit of Japanese Priority Patent Application JP2021-039637 filed on Mar. 11, 2021, and Japanese Priority Patent Application JP2022-020872 filed on Feb. 14, 2022, contents of which are incorporated herein by reference.

It should be understood by those skilled in the art that various modifications, combinations, sub-combinations, and alterations may occur depending on design requirements and other factors insofar as they are within the scope of the appended claims or the equivalents thereof.

What is claimed is:

1. A biopotential electrode body, comprising:
multiple electrically conductive portions each having a contact surface contactable with a biological body; and
one or more electrically insulating portions that electrically insulate the multiple electrically conductive portions from each other, wherein
the electrically conductive portions each include a first elastic resin and a plurality of first particles buried in the first elastic resin, and
the electrically insulating portions each include a second elastic resin and a plurality of second particles buried in the second elastic resin.

2. The biopotential electrode body according to claim 1, wherein
the first particles comprise electric conductors, and
the second particles comprise electric insulators.

3. The biopotential electrode body according to claim 2, wherein the first elastic resin and the second elastic resin comprise electric insulators.

4. The biopotential electrode body according to claim 3, wherein the first elastic resin and the second elastic resin are materials substantially identical to each other.

5. The biopotential electrode body according to claim 1, wherein the first particles include carbon particles or metal particles.

6. The biopotential electrode body according to claim 5, wherein the metal particles each include at least one of gold (Au), silver (Ag), or copper (Cu).

7. The biopotential electrode body according to claim 1, wherein the first particles each include metal particles and a metal compound, the metal compound covering at least a portion of a surface of each of the metal particles.

8. The biopotential electrode body according to claim 7, wherein the metal compound comprises a sulfide, a selenide, or a chloride of at least one metal of gold (Au), silver (Ag), or copper (Cu).

9. The biopotential electrode body according to claim 1, wherein the first particles each include a base particle and an electrically conductive organic polymer covering at least a portion of a surface of the base particle.

10. The biopotential electrode body according to claim 9, wherein the electrically conductive organic polymer includes at least one selected from a group consisting of PEDOT-PSS, polypyrrole, polyacetylene, polyphenylenevinylene, polythiophene, polythiol, polyaniline, and analogs thereof.

11. The biopotential electrode body according to claim 1, wherein the first elastic resin and the second elastic resin each comprise at least one thermoplastic resin selected from a group consisting of a polyvinylchloride resin, a polypropylene resin, a polyethylene resin, a polyurethane resin, a polyacetal resin, a polyamide resin, a polycarbonate resin, and copolymers thereof.

12. The biopotential electrode body according to claim 1, wherein the first elastic resin and the second elastic resin each comprise natural rubber or diene rubber.

13. The biopotential electrode body according to claim 1, wherein the first elastic resin and the second elastic resin each comprise a thermosetting elastomer including one selected from a group consisting of a silicone resin and a polyurethane resin.

14. The biopotential electrode body according to claim 1, wherein the second particles each include a titanium oxide.

15. The biopotential electrode body according to claim 1, wherein
the multiple electrically conductive portions and the one or more electrically insulating portions form a cylindrical structure as a whole, the cylindrical structure having a substantially cylindrical shape, and
the electrically conductive portions and the electrically insulating portions are alternately disposed in a circumferential direction of the cylindrical structure.

16. The biopotential electrode body according to claim 1, wherein
the multiple electrically conductive portions and the one or more electrically insulating portions form a cylindrical structure as a whole, the cylindrical structure having a substantially cylindrical shape, and
the multiple electrically conductive portions are separated from each other by the one or more electrically insulating portions in an axial direction of the cylindrical structure.

17. A biopotential sensor comprising a biopotential electrode body, the biopotential electrode body including:
multiple electrically conductive portions each having a contact surface contactable with a biological body; and
one or more electrically insulating portions that electrically insulate the multiple electrically conductive portions from each other, wherein
the electrically conductive portions each include a first elastic resin and a plurality of first particles buried in the first elastic resin,
the electrically insulating portions each include a second elastic resin and a plurality of second particles buried in the second elastic resin.

18. The biopotential sensor according to claim 17, wherein
the multiple electrically conductive portions include a first electrically conductive portion and a second electrically conductive portion,
the first electrically conductive portion comprises a measurement terminal that measures electric potential in the biological body, and
the second electrically conductive portion comprises a grounding terminal.

19. The biopotential sensor according to claim 17, further comprising a differential amplifier circuit, wherein
the multiple electrically conductive portions include a first electrically conductive portion and a second electrically conductive portion,
the first electrically conductive portion comprises a measurement terminal that measures electric potential in the biological body,
the second electrically conductive portion comprises a reference terminal that measures reference electric potential, and
the differential amplifier circuit detects a difference between the electric potential in the biological body and the reference electric potential.

20. The biopotential sensor according to claim 17, further comprising a determination unit that determines that the biopotential electrode body is being attached to the biological body on a basis of a biological signal acquired from the biological body.

21. The biopotential sensor according to claim 20, wherein
the multiple electrically conductive portions include a first electrically conductive portion and a second electrically conductive portion, and
the determination unit determines that the biopotential electrode body is being attached to the biological body on a basis of a change in impedance of the biological body between at the first electrically conductive portion and at the second electrically conductive portion.

22. The biopotential sensor according to claim 21, wherein the determination unit determines that the biopotential electrode body is being attached to the biological body on a basis of resistance of a measured impedance value of the biological body.

23. The biopotential sensor according to claim 21, further comprising a current supplying unit that supplies a current having a frequency of greater than 0 Hz and less than or equal to 1000 Hz between the first electrically conductive portion and the second electrically conductive portion.

24. The biopotential sensor according to claim 17, further comprising:
an oscillation circuit that causes an electrical oscillation; and
a determination unit that determines that the biopotential electrode body is being attached to the biological body on a basis of a change in oscillating frequency of the electrical oscillation.
